(19) United States
(12) Patent Application Publication
(10) Pub. No.: US 2025/0241605 A1
(43) Pub. Date: Jul. 31, 2025

(54) X-RAY DETECTOR, X-RAY DEVICE AND METHOD FOR TEMPERATURE REGULATION

(71) Applicant: Siemens Healthineers AG, Forchheim (DE)

(72) Inventors: Thomas LANGSEDER, Erlangen (DE); Stefan WOELFEL, Dormitz (DE)

(73) Assignee: Siemens Healthineers AG, Forchheim (DE)

(21) Appl. No.: 19/039,831

(22) Filed: Jan. 29, 2025

(30) Foreign Application Priority Data
Jan. 31, 2024 (DE) 10 2024 200 878.8

(51) Int. Cl. A61B 6/42; A61B 6/00

(52) U.S. Cl. CPC A61B 6/4241; A61B 6/4488

(57) ABSTRACT

One or more example embodiments relates to an X-ray detector including at least one detector unit; and a temperature regulating unit including a fluid providing unit, a heat exchanger and at least one fluid channel, the fluid providing unit configured to provide a fluid, wherein the detector unit and the temperature regulating unit are positioned in a defined arrangement relative to one another and are moveable relative to an examination object to be mapped via X-ray transirradiation, the detector unit is configured to detect X-ray radiation incident upon an X-ray sensitive surface of the detector unit, and the at least one fluid channel is arranged such that heat is transferrable between the detector unit and the fluid.

### CROSS-REFERENCE TO RELATED APPLICATION(S)

The present application claims priority under 35 U.S.C. § 119 to German Patent Application No. 10 2024 200 878.8, filed Jan. 31, 2024, the entire contents of which are incorporated herein by reference.

### FIELD

One or more example embodiments relates to an X-ray detector, an X-ray device and a method for temperature regulation of a detector unit of an X-ray detector.

### RELATED ART

Modern computed tomography (CT) devices have a gantry with a rotatable frame on which, inter alia, the X-ray source and an X-ray detector for detecting X-ray radiation are arranged. An X-ray detector of this type typically comprises an X-ray converter element which has an X-ray sensor layer and, where relevant, a layer with A/D (analog-to-digital) converters arranged thereunder. Electronic integration has recently become an important trend in X-ray converter elements. It has been an aim therein to reduce the length of the analogue path between the analogue X-ray sensor layer and the A/D converters which are usually realized as an application-specific integrated circuit (ASIC). In the case of integrating X-ray detectors, in particular, X-ray converters, the analogue X-ray sensor layer is formed by way of a suitable sensor layer, for example, a scintillator in combination with a photodiode, whereas in counting X-ray converters, in particular, photon-counting X-ray converters, a direct-converting semiconductor sensor is used. An A/D converter configured as an ASIC then generates the digital output signal. In both cases, an integration of the ASICs into a compact structure, in particular, a stacked structure, together with the analogue X-ray sensor layer, brings a significant heat source closer to the X-ray sensor elements of the X-ray sensor layer itself.

Since the X-ray sensor elements react very sensitively to temperature variations, heat management is a critical task in the development of a modern X-ray detector. Therein, particular challenges of heat management are keeping the operating temperature of the X-ray detector stable, preventing temperature gradients between neighboring X-ray converter elements and also reducing temperature gradients within each X-ray converter element. These challenges are even more important in counting X-ray detectors since the directly converting semiconductor sensors are additional heat sources and their sensor performance, for example, a stability of a counting rate, also reacts highly sensitively to thermal changes.

Due to temporally changing thermal boundary conditions, for example, the temperature, an air humidity and/or an air volume flow rate, for the, in particular, photon-counting X-ray detector, artifacts can occur in the generated image, for example, a dark or light central point and/or rings. As a result, it is often difficult to achieve an adequate image quality with a photon-counting CT system.

Currently, for temperature regulation of the X-ray detector, in particular, of X-ray detector modules and other components of the gantry, a central heat-exchanger with a central pressure channel is often used for all the rotating components. Almost every component has the possibility to place a demand on this central regulator, with the result that different temperatures and airflows are generated. These can vary in the range of 19-27° C. and a flow rate of 600-3000 m3/h. The X-ray detector is therein often per se open and the air circulates in the whole room.

### SUMMARY

In order to counteract thermal cooling variations, for example, a heating unit which evens out cooling variations close to the X-ray detector is needed. The heat output of the heating unit is, however, often limited to the rotating part of the gantry. In addition, a control of the heating unit requires a high-frequency readout of the current temperature of the X-ray detector in order to be able to react rapidly to short-term cooling changes.

One or more example embodiments enables an efficient and stable temperature regulation of an X-ray detector.

The is achieved at least by way of the subject matter of the independent claims. Advantageous embodiments with suitable developments are the subject matter of the subclaims. Independent of the grammatical term usage, individuals with male, female or other gender identities are included within the term.

### BRIEF DESCRIPTION OF THE DRAWINGS

Exemplary embodiments of the invention are illustrated in the drawings and are described in greater detail below. In the different figures, the same reference signs are used for the same features. In the drawings:

FIGS. 1 to 5 show schematic representations of different advantageous embodiments of an X-ray detector,

FIGS. 6 and 7 show schematic representations of different advantageous embodiments of a proposed X-ray device,

FIGS. 8 and 9 show schematic representations of different advantageous embodiments of a method for temperature regulation of a detector unit of an X-ray detector.

### DETAILED DESCRIPTION

One or more example embodiments relates in a first aspect to an X-ray detector comprising at least one detector unit and a temperature regulating unit. The detector unit and the temperature regulating unit are positioned in a defined arrangement relative to one another and able to be moved in relation to an examination object to be mapped via X-ray transirradiation. The detector unit is configured for detecting X-ray radiation incident upon an X-ray sensitive surface of the detector unit. The temperature regulating unit has a fluid providing unit, a heat exchanger and at least one fluid channel. In addition, the fluid providing unit is configured for providing a fluid. The heat exchanger is configured for temperature-regulating the fluid to a pre-defined temperature or a pre-defined temperature range. Furthermore, the provided temperature-regulated fluid can flow through the at least one fluid channel. In addition, the at least one fluid channel is arranged such that heat can be transferred between the detector unit and the fluid.

Advantageously, the detector unit and the temperature regulating unit are arranged in a defined, in particular, a fixed arrangement relative to one another. In addition, the detector unit and the temperature regulating unit, in particular, the defined arrangement of the detector unit and the temperature regulating unit are movable, for example, able to be rotated and/or translated relative to the examination object that is to be mapped via X-ray transirradiation. For example, the defined arrangement of the detector unit and the temperature regulating unit can be arranged on a movable part of an X-ray device, for example, a C-arm or a rotor of a gantry, in particular, integrated in the C-arm or the rotor.

The detector unit can have an X-ray sensitive surface, in particular, an X-ray detector layer, on its upper side. In an operating state of the X-ray detector, it can face toward an X-ray source. Furthermore the X-ray detector layer can be configured for detecting X-rays emitted by the X-ray source. The X-ray detector layer can comprise a direct-converting (semiconductor) X-ray sensor layer, for example having CdTe, CdZnTe, CdTeSe, CdZnTeSe, CdMnTe, GaAs, Si or Ge as the semiconductor material. The X-ray detector layer can also comprise an X-ray sensor layer which is configured for converting X-ray radiation into light and optically coupled photodiodes, in particular, one or more photodiode arrays. As the material, scintillator material, for example GOS (Gd2O2S), CsJ, YGO or LuTAG is often utilized. The X-ray detector layer can also comprise a layer with analogue-to-digital converters onto which the X-ray sensor layer is applied, wherein the A/D converter layer can be realized in one or more ASICs. The X-ray detector layer can be applied on a base plate, also referred to as material, for example, a conductor panel or a ceramic or glass material which then forms the underside of the detector unit.

Advantageously, the detector unit, in particular, a thermal contact surface on the underside of the detector unit, can be in thermal contact with the, in particular, metallic, material which surrounds the hollow space of the at least one fluid channel. By this means, the heat can be transferred between the detector unit and the fluid which, in the operating state, flows through the at least one fluid channel. In particular, the detector unit and the at least one fluid channel can be arranged stacked along an X-ray incidence direction. The detector unit can have an, in particular, metallic thermal contact surface on its underside. The heat-conducting contact can be further improved by, for example, heat-conducting pastes or pads or adhesives, in particular, heat-conducting adhesives or solder materials.

The temperature regulating unit has the fluid providing unit. The fluid providing unit is configured for providing the fluid. In particular, the fluid providing unit can be configured for providing the fluid to the at least one fluid channel, in particular, the plurality of fluid channels. By way of example, in an operating state of the X-ray detector, the fluid providing unit can provide the fluid to an opening of the at least one fluid channel, in particular, to an opening of each of the plurality of fluid channels. The fluid providing unit can comprise, for example, a pump and/or a blower and/or nozzle.

Advantageously, the fluid providing unit can be configured for providing the fluid via the heat exchanger to the at least one fluid channel. In particular, the fluid providing unit can be configured for providing the fluid to the heat exchanger. The heat exchanger can be configured for transferring heat, in particular, thermal energy, from the fluid to a medium, for example, another fluid.

In this way, the heat exchanger is configured for temperature-regulating the fluid to the pre-defined temperature or a pre-defined temperature range. In particular, the heat exchanger can be configured for providing the fluid provided by the fluid providing unit, having the pre-defined temperature or a temperature within the pre-defined temperature range, to the at least one fluid channel.

The temperature regulating unit has at least one fluid channel, in particular, a plurality of fluid channels. The at least one fluid channel can be configured as a tunnel-shaped hollow space in an, in particular, metallic material. The at least one fluid channel can be configured to have the fluid, for example a liquid, in particular, water and/or a gas and/or a gas mixture, in particular, air, flow through it. Preferably, for fluid-based temperature regulation of the detector unit, air is used as the fluid. Therein, in an operating state of the X-ray detector, the fluid can flow through the hollow space of the at least one fluid channel. Furthermore, the hollow spaces can be configured for conducting, in particular, for transporting, the fluid.

The at least one fluid channel can advantageously be capable of having the provided temperature-regulated fluid flow through it. In particular, in an operating state of the X-ray detector, the at least one fluid channel can have the temperature-regulated fluid flow through it. If the temperature regulating unit has a plurality of fluid channels, in the operating state of the X-ray detector, the fluid channels can thus have the temperature-regulated fluid flow through them, in particular, simultaneously. The at least one fluid channel can be configured, for example, as a pressure channel.

The at least one fluid channel, in particular, the plurality of fluid channels, can be arranged, in particular, positioned such that it is possible for heat to be transferred between the detector unit and the fluid. Advantageously, the detector unit, in particular, the thermal contact surface on the underside of the detector unit, can be in thermal contact with the, in particular, metallic, material which surrounds the hollow space of the at least one fluid channel. By this means, the heat can be transferred between the detector unit and the fluid which, in the operating state, flows through the at least one fluid channel. In particular, the detector unit and the at least one fluid channel can be arranged stacked along an X-ray incidence direction. In addition, a volume and/or an aperture size of the at least one fluid channel and/or a fluid quantity provided by the fluid providing unit for the temperature regulation of the fluid can be adjusted to the pre-defined temperature range.

The proposed embodiment of the X-ray detector can advantageously enable an efficient and stable temperature regulation of the X-ray detector. By way of the movable mounting of the X-ray detector and the temperature regulating unit in the defined arrangement, a rotation-independent temperature regulation of the X-ray detector can also be enabled. By this means, tuning steps, in particular, rotation-dependent conditioning of the X-ray detector can advantageously be reduced.

In a further advantageous embodiment of the proposed X-ray detector, the detector unit can be configured for photon-counting detection of the X-ray radiation incident upon the X-ray sensitive surface of the detector unit.

The X-ray detector can advantageously have a plurality of photon-counting detector elements which can be arranged in the form of rows, columns and/or a grid. The detector elements can each be configured for counting X-ray photons that are emitted by the X-ray source during the X-ray transirradiation. For this purpose, the detector elements can each have a number of pixels, wherein, in each case, the X-ray photons incident upon the pixels can be counted. Therein, per pixel, different energies can be distinguished by each of the detector elements, for example four different energies of X-ray photons. The differentiation of the energies, in particular, the photon energies can take place, for example, by allocating the energies of the captured X-ray photons to four intervals. For each detector element, for example, four different energy levels can be distinguished, that is, the X-ray photons falling into the energy intervals resulting therefrom can be separately counted.

For this purpose, the detector elements can each receive physical signals, for example, X-ray photons, wherein corresponding data describes, for example, the X-ray photons counted per pixel of the detector element and/or the X-ray photons counted for each of the different energy intervals. The data acquired by the detector elements can be transferred to an electric circuit or can be read out from the detector elements by it. In order to undertake a further processing of the data recorded by the detector elements, the electric circuit can comprise a storage facility with at least one storage block. The data generated by the detector elements can be stored, at least temporarily, in the storage block. The individual detector elements can preferably be configured as special, application-specific integrated circuits (ASICs). On the basis of the X-ray photons counted via the detector elements over a pre-determined detection period, a dose value per detector element, in particular, a dose value per pixel, can be determined.

The proposed embodiment can facilitate an improved, in particular, stable and high, image quality via the efficient and stable temperature regulation of the X-ray detector.

In a further advantageous embodiment of the proposed X-ray detector, the temperature regulating unit can further comprise a heating element. Therein, the heating element can be configured for heating the detector unit to a further pre-defined temperature.

The further pre-defined temperature can constitute an operating temperature of the detector unit. The further pre-defined temperature can be, for example, between 30° C. and 45° C. The heating element can advantageously comprise a heating wire for electrically heating the detector unit. Alternatively or additionally, the heating element can comprise a light source for optically heating the detector unit, for example, via infrared light. Advantageously, the heating element can be arranged on the detector unit or integrated into the detector unit. Alternatively, the heating element can be arranged spaced from the detector unit, for example in a configuration of the heating element as a light source for optically heating the detector unit. If the detector unit comprises a plurality of detector elements, the temperature regulating unit can advantageously comprise a plurality of heating elements, in particular, a heating element for each of the detector elements. Therein, the heating elements can be configured for heating the respective detector element to the respective further pre-defined temperature.

Via the heating element, the proposed embodiment can enable a particularly rapid equalization of temperature variations in the detector unit. Advantageously, temperature-regulated fluid can temperature-regulate the detector unit at least roughly, while the heating element can enable a fine adjustment of the temperature of the detector unit. In this way, a smaller heating power buffer can be provided since, with the same cooling effect of the fluid, the detector unit can be operated with a constant heating power level from the at least one heating element.

In a further advantageous embodiment of the proposed X-ray detector, the fluid can comprise a gas mixture. In addition, the temperature regulating unit can comprise a dehumidifier which is configured for adjusting a water content in the fluid before it flows through the at least one fluid channel, in such a way that a dew point temperature of the fluid lies below a current temperature of the fluid.

Advantageously, the fluid can comprise a gas mixture, for example, air. The fluid, in particular, the gas mixture, can comprise water vapor. Advantageously, the temperature regulating unit can comprise a dehumidifier, in particular, a condensation dryer and/or an adsorption dryer. Advantageously, the fluid providing unit can be configured for providing the fluid to the heat exchanger or the dehumidifier. According to a first variant, the heat exchanger can be configured for providing the temperature-regulated fluid to the dehumidifier. Alternatively, the dehumidifier can be configured for providing the fluid to the heat exchanger.

The dehumidifier can be configured for adjusting, in particular, for reducing, the water content in the fluid. In particular, the dehumidifier can be configured for adjusting the water content in the fluid in such a way that the water content lies below a pre-determined threshold value. Advantageously, the dehumidifier can be configured for adjusting the water content in the fluid in such a way that a dew point of the fluid lies below a current temperature of the, in particular, temperature-regulated fluid. In this way, it can advantageously be ensured that no condensation of the water vapor contained in the fluid takes place within the at least one fluid channel.

A condensate, in particular, water, forming in the dehumidifier during the dehumidifying can advantageously be collected at least temporarily in a container or can be conducted away via a conduit.

By this means, the fluid can be provided to the at least one fluid channel in a stable manner with regard to temperature and air humidity.

In a further advantageous embodiment of the proposed X-ray detector, the fluid can comprise a gas or a gas mixture. In addition, the fluid providing unit can be configured for providing the fluid from a first environment to the heat exchanger. Furthermore, the temperature regulating unit can be configured for discharging the fluid after an exchange of heat with the detector unit, from the at least one fluid channel into a second environment.

The fluid can comprise a gas or a gas mixture, for example, air. The first environment can be a first spatial region, in particular, a first volume which adjoins the fluid providing unit and/or at least partially surrounds the fluid providing unit. The first environment can be enclosed, for example, surrounded by a wall, or open, in particular, at most partially surrounded by a wall. Therein, in a first operating state of the X-ray detector, the first environment can comprise, in particular, have available, the gas or gas mixture. The fluid providing unit can be configured for providing the fluid, in particular, the gas or gas mixture from the first environment to the heat exchanger. For this purpose, the fluid providing unit can be configured for sucking the fluid from the first environment, in particular, continually, for example, via a suction aperture. In addition, the fluid providing unit can be configured for providing the sucked-in fluid to the heat exchanger. In order to suck in the fluid and to provide the fluid to the heat exchanger, the fluid providing unit can comprise, for example, a blower and/or a propeller and/or a pump and/or a nozzle.

Furthermore, the temperature regulating unit can be configured for discharging the fluid after the exchange of heat with the detector unit, from the at least one fluid channel, in particular, the plurality of fluid channels, into a second environment, in particular, continuously, for example, via at least one outlet aperture to the at least one fluid channel.

The second environment can be a second spatial region, in particular, a second volume which adjoins the temperature regulating unit and/or at least partially surrounds the temperature regulating unit. The second environment can be enclosed, for example, surrounded by a wall, or open, in particular, at most partially surrounded by a wall.

Advantageously, the first and the second environment can be spaced from one another, at least partially coincide or adjoin one another. The first and the second environment can, in particular, identify different spatial regions of a common volume. According to a first variant, the fluid providing unit can suck in the fluid from the first environment and provide it to the heat exchanger. Furthermore, the temperature regulating unit can discharge the fluid after the completed exchange of heat with the detector unit, from the at least one fluid channel into the second environment. The fluids in the first and the second environment can diffuse. Alternatively, on the basis of a spatial separation between the first and the second environment, it can be ensured that no repeated provision of the fluid takes place, in particular, that a diffusion between the fluid in the first and the second environment does not take place.

Therein, the first environment, in particular, a spatial extent of the first environment can be defined by way of a suction power of the fluid providing unit for sucking in the fluid. Similarly, the second environment, in particular, a spatial extent of the second environment can be defined by way of an extent of the at least one fluid channel on the discharge of the heated fluid.

The proposed embodiment can enable a simple provision of the fluid from the first environment. In addition, by this means, further components of the temperature regulating unit for conducting or storing the fluid can be dispensed with.

In a further advantageous embodiment of the proposed X-ray detector, the fluid providing unit can be configured for providing the fluid, after an exchange of heat with the detector unit, from the at least one fluid channel repeatedly to the heat exchanger.

The temperature regulating unit can be configured for providing the fluid, after the exchange of heat with the detector unit, from the at least one fluid channel, in particular, the plurality of fluid channels, via at least one outlet aperture to the at least one fluid channel. Advantageously, the X-ray detector can have at least one further fluid channel which connects the at least one outlet aperture to an inlet aperture of the fluid providing unit, in particular, in a fluid-tight manner. The at least one further fluid channel can be configured for providing the fluid provided from the outlet aperture to the inlet aperture of the fluid providing unit. In particular, the fluid providing unit can be configured for sucking in the fluid provided via the at least one further fluid channel to the inlet aperture and to provide it to the heat exchanger. By this means, the fluid heated during the exchange of heat with the detector unit can be temperature-regulated repeatedly via the heat exchanger to the pre-defined temperature or the pre-defined temperature range and provided to the at least one fluid channel. By this means, the fluid can circulate in a closed circuit.

The proposed embodiment can advantageously minimize variations in a temperature of the fluid provided to the heat exchanger. By this means, the temperature regulation of the X-ray detector can take place still more efficiently and more stably.

In a further advantageous embodiment of the proposed X-ray detector, the fluid providing unit can comprise a pump and/or a blower and/or a nozzle. Therein, the pump and/or the blower and/or the nozzle can be configured for providing the fluid at a pre-defined pressure.

The pump and/or the blower and/or the nozzle can be configured for sucking in the fluid. In addition, the pump and/or the blower and/or the nozzle can be configured for providing the fluid at a pre-defined pressure, in particular, a positive pressure relative to an ambient pressure, to the heat exchanger and/or the at least one fluid channel.

The proposed embodiment can advantageously ensure a defined flow rate of the fluid through the at least one fluid channel.

In a further advantageous embodiment of the proposed X-ray detector, the detector unit can comprise a plurality of detector modules. Therein, the temperature regulating unit can comprise at least one fluid channel to each of the plurality of detector modules. Furthermore, the fluid channels can have the fluid flow through them. In addition, the fluid channels can be arranged such that heat can be transferred between the detector modules and the fluid.

Advantageously, the detector unit can comprise a plurality of detector modules which are arranged in rows, in columns and/or in a grid form. The detector modules can be configured at least partially identically or differently. Advantageously, the temperature regulating unit can comprise at least one, in particular, exactly one, fluid channel to each of the plurality of detector modules. Therein, the plurality of fluid channels can have the fluid flow through them, in particular, simultaneously. The plurality of fluid channels can be arranged separated from one another along a longitudinal extension direction. Alternatively, at least a subset of the plurality of fluid channels can have a connection along a respective longitudinal extension direction to at least one further of the plurality of fluid channels. Advantageously, the heat exchanger can be configured for providing the temperature-regulated fluid to the plurality of fluid channels, in particular, simultaneously. Advantageously, in an operating state of the X-ray detector, the temperature-regulated fluid flows through the plurality of fluid channels. Therein, the plurality of fluid channels can be arranged such that heat can be transferred between the detector modules and the fluid.

Therein, the plurality of fluid channels can be arranged such that heat can be transferred between the detector modules and the fluid. Advantageously, the plurality of detector modules, in particular, a thermal contact surface on an underside of each detector module, can be in thermal contact with the, in particular, metallic material which surrounds the hollow space of the at least one fluid channel of each of the plurality of fluid channels. By this means, the heat can be transferred between the detector modules and the fluid which, in the operating state, flows through the at least one fluid channel of each of the plurality of fluid channels. In particular, the detector modules and the at least one corresponding of the plurality of fluid channels can be arranged stacked along an X-ray incidence direction.

The proposed embodiment can advantageously enable an efficient and stable temperature regulation of the plurality of detector modules.

In a further advantageous embodiment of the proposed X-ray detector, the fluid providing unit to each of the detector modules can comprise a subsidiary fluid providing unit which is configured for providing the temperature-regulated fluid to the respective fluid channels.

The plurality of subsidiary fluid providing units can be configured at least partially identically or differently. The plurality of subsidiary fluid providing units can each be arranged between the heat exchanger and a respective first aperture of the plurality of fluid channels. For this purpose, the subsidiary fluid providing units can be configured for providing the fluid temperature-regulated via the heat exchanger via each first aperture to the respective fluid channel, for example, via a positive pressure. Alternatively, the fluid channels can be arranged between the heat exchanger and the subsidiary fluid providing units. For this purpose, the subsidiary fluid providing units can be configured for sucking the temperature-regulated fluid provided via the heat exchanger via each first aperture of the plurality of fluid channels, via each second aperture of the fluid channels, for example, via a negative pressure.

The proposed embodiment can advantageously enable a dedicated temperature regulation of the plurality of detector modules. In particular, by this means, fluid volume differences, in particular, air volume differences between the detector modules can be equalized.

In a further advantageous embodiment of the proposed X-ray detector, the X-ray detector can further comprise a sensor which is configured for capturing a current temperature of the fluid and/or of the detector unit and/or a currently provided fluid quantity. Furthermore, the heat exchanger can be configured for temperature-regulating the fluid dependent upon the current temperature and/or the currently provided fluid quantity. Alternatively or additionally, the fluid providing unit can be configured for adjusting a provided fluid quantity per unit time dependent upon the current temperature.

The sensor can comprise an, in particular, optical and/or electromagnetic and/or mechanical and/or chemical temperature sensor. Advantageously, the temperature sensor can be configured for capturing the current temperature of the fluid, in particular, before the provision to the heat exchanger and/or the current temperature of the detector unit, in particular, the detector modules. In particular, the temperature sensor can be configured for providing a signal dependent upon the captured current temperature of the fluid and/or the detector unit. The temperature sensor can advantageously be arranged at least partially on or in a region through which, in an operating state of the X-ray detector, the fluid flows.

The sensor can further comprise an, in particular, optical and/or electromagnetic and/or mechanical fluid quantity sensor, for example, an air flow meter. Advantageously, the fluid quantity sensor can be configured for capturing the fluid quantity currently provided by the fluid providing unit to the heat exchanger, in particular, a fluid volume per unit time. Furthermore, the fluid quantity sensor can be configured for providing a signal dependent upon the captured currently provided fluid quantity. The fluid quantity sensor can advantageously be arranged at least partially on or in a region through which, in an operating state of the X-ray detector, the fluid flows after its provision by the fluid providing unit.

The heat exchanger can be configured for temperature-regulating the fluid dependent upon the current temperature, in particular, on the basis of the signal provided by the temperature sensor, and/or on the basis of the currently provided fluid quantity, in particular, on the basis of the signal provided by the fluid quantity sensor. In particular, the heat exchanger can be configured for adjusting the pre-defined temperature or the pre-defined temperature range to temperature-regulate the fluid dependent upon the current temperature and/or the currently provided fluid quantity. By this means, a pre-defined temperature difference between the temperature-regulated fluid and the current temperature can be ensured. In addition, a sufficient exchange of heat between the detector unit and the fluid for temperature regulation of the detector unit can be ensured.

Alternatively or additionally, the fluid providing unit can be configured for adjusting the fluid quantity provided, in particular, a fluid volume provided per unit time, in particular, per time interval, dependent upon the current temperature. For example, the fluid providing unit can be configured for increasing the fluid quantity provided per unit time on identification of a rise in the current temperature and, on identification of a fall in the current temperature, for decreasing it.

The proposed embodiment can advantageously enable a precise temperature regulation of the X-ray detector, in particular, the detector unit.

In a further advantageous embodiment of the proposed X-ray detector, the subsidiary fluid providing units can be configured for adjusting a respectively provided fluid quantity per unit time dependent upon the current temperature individually and/or in a coordinated manner.

Advantageously, the sensor, in particular, the temperature sensor can be configured for capturing the current temperature of the fluid in the plurality of fluid channels individually. Alternatively or additionally, the sensor can be configured for capturing the current temperature of the plurality of detector modules individually. Furthermore, the plurality of subsidiary fluid providing units can be configured for adjusting the respectively provided fluid quantity, in particular, the respectively provided fluid volume per unit time, in particular, per time interval, dependent upon the current temperatures captured, individually and/or in a coordinated manner.

For example, the subsidiary fluid providing units can be configured for increasing the respectively provided fluid quantity per unit time on identification of a rise, in particular, in the respective current temperature, in particular, individually and/or in a coordinated manner and, on identification of a fall, in particular, in the respective current temperature, for decreasing it, in particular, individually and/or in a coordinated manner. According to an embodiment, the subsidiary fluid providing units can be configured for adjusting the respectively provided fluid quantity per unit time dependent upon the current temperature in a coordinated manner such that a minimum fluid quantity per unit time is not exceeded in any of the plurality of fluid channels.

The proposed embodiment can advantageously enable a precise temperature regulation of the plurality of detector modules.

In a further advantageous embodiment of the proposed X-ray detector, the heat exchanger can be configured for temperature-regulating the fluid to a temperature between 18° C. and 30° C.

Advantageously, the heat exchanger can be configured for temperature-regulating the fluid to a temperature between 18° C. and 30° C. In particular, the heat exchanger can be configured for providing the fluid at a temperature of between 18° C. and 30° C. to the at least one fluid channel. This temperature range often corresponds to a typical ambient temperature of the X-ray detector. It can thereby advantageously be achieved that the heat exchanger is not required to generate any large temperature differences. In addition, a volume and/or an aperture size of the at least one fluid channel and/or a fluid quantity provided by the fluid providing unit for the temperature regulation of the fluid can be adjusted to the pre-defined temperature range from 18° C. to 30° C.

One or more example embodiments relates to an X-ray device comprising an X-ray source and a proposed X-ray detector. Therein, the X-ray source is configured for emitting X-ray radiation for transilluminating an examination object arranged between the X-ray source and the X-ray detector.

Advantageously, the medical X-ray device can be configured as a computed tomography (CT) system and/or as a C-arm X-ray device and/or an O-arm X-ray device.

The X-ray detector can be configured for detecting the X-ray radiation emitted by the X-ray source, in particular, following an interaction with the examination object.

The advantages of the proposed X-ray device substantially correspond to the advantages of the proposed X-ray detector. Features, advantages or alternative embodiments mentioned herein can also be transferred similarly to the other claimed subject matter and vice versa.

In a further advantageous embodiment of the proposed X-ray device, the X-ray source and the X-ray detector can be mounted in a defined arrangement able to rotate about a common rotation axis.

The X-ray device can advantageously comprise a gantry with a rotor. The gantry can comprise an annular structure, in particular, a stator, and the rotor. The X-ray source and the X-ray detector, in particular, the defined arrangement of the detector unit and the temperature regulating unit can be arranged in a defined arrangement on the rotor, in particular, integrated into the rotor or fastened onto the rotor. The rotor can be mounted able to rotate, in particular, relative to the stator. Therein, an examination object that is to be mapped can be capable of being arranged within an opening of the gantry, in particular, between the X-ray source and the X-ray detector, for X-ray transirradiation.

One or more example embodiments relates to a method for temperature regulation of a detector unit of a proposed X-ray detector. In a first step, the fluid is provided via the fluid providing unit to flow through the at least one fluid channel. Therein, heat is transferred between the detector unit and the fluid. In a further step, a current temperature of the fluid and/or of the detector unit and/or a currently provided fluid quantity is captured via a sensor. In dependence upon the captured current temperature and/or captured currently provided fluid quantity, the heat exchanger is controlled such that the fluid is temperature-regulated to a pre-defined temperature or a pre-defined temperature range. Alternatively or additionally, the fluid providing unit is controlled dependent upon the captured current temperature such that a fluid quantity provided per unit time is adjusted.

The advantages of the proposed method substantially correspond to the advantages of the proposed X-ray detector. Features, advantages or alternative embodiments mentioned herein can also be transferred similarly to the other claimed subject matter and vice versa.

An algorithm for controlling the heat exchanger and/or the fluid providing unit, in particular, the plurality of subsidiary fluid providing units, on the basis of simulations and/or temperature measurements can be determined, in particular, adjusted, in particular, for different recording scenarios of the X-ray detector. Furthermore, the algorithm can be provided on a data measurement system (DMS) of the X-ray detector, for example, in the context of providing a correction algorithm for correcting known defective detector pixels. By this means, a tuning time of the X-ray detector can advantageously be minimized.

In a further advantageous embodiment of the proposed method, the fluid can comprise a gas mixture. Therein, a water content in the fluid can be adjusted via a dehumidifier before it flows through the at least one fluid channel, in such a way that a dew point temperature of the fluid lies below a current temperature of the fluid.

One or more example embodiments relates to a computer program product with a computer program which can be loaded directly into a memory store of a processing unit, having program portions in order to carry out all the steps of a proposed method for temperature regulation of a detector unit of a proposed X-ray detector when the program portions are executed by the processing unit. The computer program product can therein comprise an item of software with a source code which must still be compiled and linked or which must only be interpreted, or an executable software code which, for execution, need only be loaded into the processing unit. Via the computer program product, the method for temperature regulation of a detector unit of a proposed X-ray detector can be carried out rapidly, exactly reproducibly and robustly via a processing unit. The computer program product is configured such that it can carry out the method steps according to one or more example embodiments via the processing unit.

The computer program product is stored, for example, on a computer-readable storage medium or is deposited on a network or server from where it can be loaded into the processor of a processing unit which can be directly connected to the providing unit, or which can be configured as part of the providing unit. Furthermore, control information of the computer program product can be stored on an electronically readable data carrier. The items of control information of the electronically readable data carrier can be configured such that it carries out a method according to one or more example embodiments when the data carrier is used in a processing unit. Examples of electronically readable data carriers are a DVD, a magnetic tape or a USB stick, on which electronically readable control information, in particular, software, is stored. If this control information is read from the data carrier and stored in a processing unit, all the embodiments according to one or more example embodiments of the above-described methods can be carried out.

A realization largely through software has the advantage that conventionally used processing units can also easily be upgraded with a software update in order to operate in the manner according to one or more example embodiments. Such a computer program product can comprise, where relevant, in addition to the computer program, additional constituents, such as, for example, documentation and/or additional components as well as hardware components, for example, hardware keys (dongles, etc.) in order to use the software.

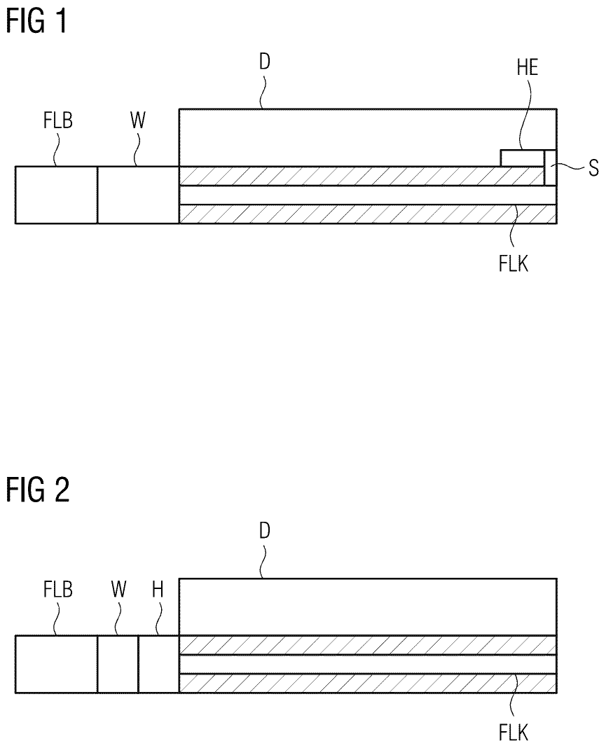

FIG. 1 shows a schematic representation of an advantageous embodiment of a proposed X-ray detector. The X-ray detector can comprise at least one detector unit D and a temperature regulating unit. The detector unit D and the temperature regulating unit can be positioned in a defined arrangement relative to one another and able to be moved relative to an examination object that is to be mapped via X-ray transirradiation. Furthermore, the detector unit D is configured for detecting X-ray radiation incident upon an X-ray sensitive surface of the detector unit D. The temperature regulating unit can have a fluid providing unit FLB, a heat exchanger W and at least one fluid channel FLK. Therein, the fluid providing unit FLB can be configured for providing a fluid. In addition, the heat exchanger W can be configured for temperature-regulating the fluid to a pre-defined temperature or a pre-defined temperature range. In particular, the heat exchanger W can be configured for temperature-regulating the fluid to a temperature between 18° C. and 30° C. In addition, the at least one fluid channel FLK can be capable of having the provided temperature-regulated fluid flow through it. The at least one fluid channel FLK can be arranged such that heat can be transferred between the detector unit D and the fluid.

Advantageously, the detector unit D can be configured for photon-counting detection of the X-ray radiation incident upon the X-ray sensitive surface of the detector unit D.

The fluid providing unit FLB can be or comprise a pump and/or a blower and/or nozzle. Therein, the pump and/or the blower and/or the nozzle can be configured for providing the fluid at a pre-defined pressure.

The fluid can comprise a gas or a gas mixture. Therein, the fluid providing unit FLB can be configured for providing the fluid from a first environment to the heat exchanger W. Furthermore, the temperature regulating unit can be configured for discharging the fluid, after an exchange of heat with the detector unit D, from the at least one fluid channel FLK into a second environment.

The X-ray detector can further comprise a sensor S which is configured for capturing a current temperature of the fluid and/or of the detector unit and/or a currently provided fluid quantity. Therein, the heat exchanger W can be configured for temperature-regulating the fluid dependent upon the current temperature and/or the currently provided fluid quantity. Alternatively or additionally, the fluid providing unit FLB can be configured for adjusting a provided fluid quantity per unit time dependent upon the current temperature.

Advantageously, the temperature regulating unit can further comprise a heating element HE. Therein, the heating element HE can be configured for heating the detector unit to a further pre-defined temperature.

The X-ray detector can comprise, for example, a processing unit (not shown here) or can be coupled to the processing unit for signal purposes. Therein, the processing unit can be configured for controlling the temperature regulating unit, in particular, the fluid providing unit FLB and/or the heat exchanger W and/or the heating element HE, in particular, dependent upon the current temperature. For this purpose, the sensor S can also be coupled for signal purposes to the processing unit.

FIG. 2 shows a schematic representation of a further advantageous embodiment of a proposed X-ray detector. Advantageously, the fluid can comprise a gas mixture. In addition, the temperature regulating unit can comprise a dehumidifier H which is configured for adjusting a water content in the fluid before it flows through the at least one fluid channel FLK, such that a dew point temperature of the fluid lies below a current temperature of the fluid.

Advantageously, the processing unit (not shown here) can further be configured for controlling the dehumidifier H, in particular, dependent upon the current temperature.

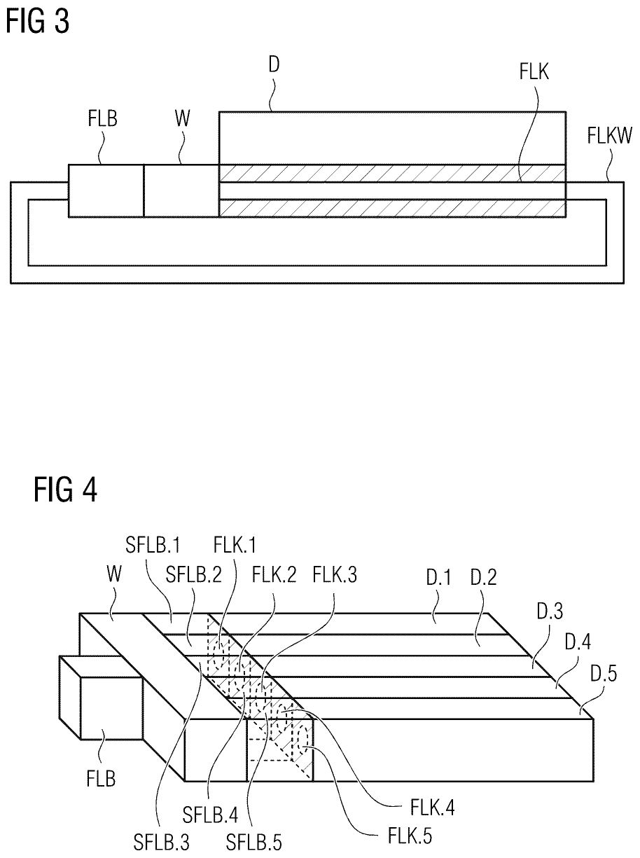

FIG. 3 shows a schematic representation of a further advantageous embodiment of a proposed X-ray detector. Therein, the fluid providing unit can be configured for providing the fluid, following an exchange of heat with the detector unit, from the at least one fluid channel repeatedly to the heat exchanger.

FIG. 4 shows a schematic representation of a further advantageous embodiment of a proposed X-ray detector. Therein, the detector unit D can comprise a plurality of detector modules D.1 to D.5. The temperature regulating unit can further comprise at least one fluid channel FLK.1 to FLK.5 to each of the plurality of detector modules D.1 to D.5. Therein, the fluid channels FLK.1 to FLK. 5 can have the fluid flow through them. In addition, the fluid channels FLK.1 to FLK. 5 can be arranged such that heat can be transferred between the detector modules D.1 to D.5 and the fluid. Advantageously, the fluid providing unit FLB to each of the detector modules D.1 to D.5 can comprise a subsidiary fluid providing unit SFLB.1 to SFLB. 5 which is configured for providing the temperature-regulated fluid to the respective fluid channels FLK.1 to FLK.5.

Advantageously, the subsidiary fluid providing units SFLB.1 to SFLB.5 can be configured for adjusting a respectively provided fluid quantity per unit time, dependent upon the current temperature, individually and/or in a coordinated manner.

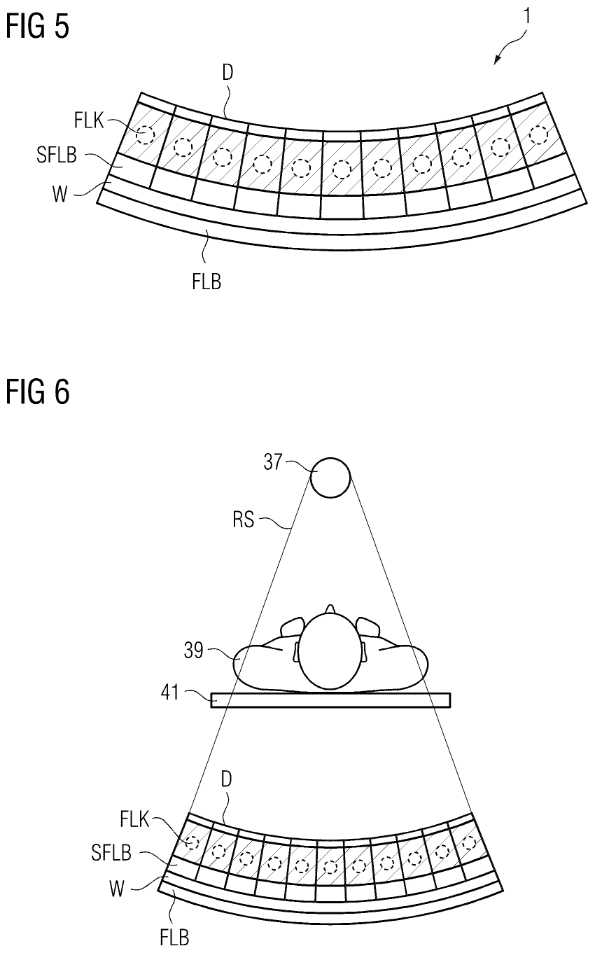

FIG. 5 shows a schematic representation of a further advantageous embodiment of a proposed X-ray detector 1. Therein, the plurality of detector modules D.i of the detector unit D can be arranged in a row, in particular, curved, for example concavely, in relation to a radiation incidence direction. Therein, i denotes an index which increases from 1 to a number of the detector modules, for example, 11 detector modules, as shown in FIG. 5. Furthermore, the detector modules D.i, the respective fluid channels FLK.i and/or the respective subsidiary fluid providing units SFLB.i are arranged stacked, in particular, along the radiation incidence direction. Therein, in FIGS. 5 and 6, for the sake of clarity, the indices i are not shown in the reference signs of the plurality of detector modules D.i of the plurality of fluid channels FLK.i and the plurality of subsidiary fluid providing units SFLB.i.

FIG. 6 shows a schematic representation of an advantageous embodiment of a proposed X-ray device. The X-ray device can comprise an X-ray source 37 and a proposed X-ray detector 1. The X-ray source 37 can be configured for emitting X-ray radiation RS for transilluminating an examination object 39 arranged between the X-ray source 37 and the X-ray detector 1, in particular, on a patient positioning apparatus 41. Advantageously, the X-ray source 37 and the X-ray detector 1 can be mounted in a defined arrangement able to rotate about a common rotation axis.

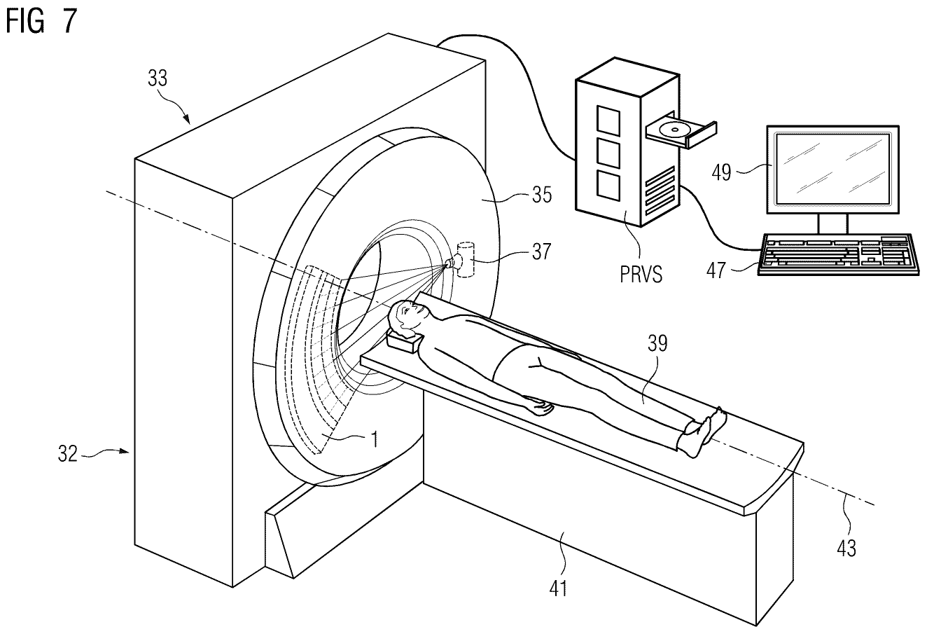

FIG. 7 shows a schematic representation of a further advantageous embodiment of a proposed X-ray device as a medical CT device 33. The CT device 33 can comprise the X-ray source 37, the X-ray detector 1 and a processing unit PRVS. Therein, the X-ray source 37 and the X-ray detector 1 can be arranged opposite one another. The X-ray source 37 can be designed to irradiate the X-ray detector 1 with X-ray radiation along an X-ray incidence direction. The X-ray detector 1 can comprise a direct-converting (semiconductor) X-ray detector layer. Therein, the X-ray detector layer can have, for example CdTe, CdZnTe, CdTeSe, CdZnTeSe or CdMnTe as the semiconductor material. The X-ray detector layer can also comprise a layer with analogue-to-digital converters onto which the X-ray sensor layer is applied, wherein the A/D converter layer can be realized in one or more ASICs.

The CT device 33 can also have a gantry 32 with a rotor 35. The X-ray source 37 and the X-ray detector 1 can be arranged in a defined arrangement on the rotor 35, in particular, integrated into the rotor 35 or fastened onto the rotor 35. The rotor 35 can be mounted able to rotate about a rotation axis 43. The examination object 39 to be mapped can be mounted on the patient positioning apparatus 41 and can be moved along the rotation axis 43 through the gantry 32. The processing unit PRVS can be used for controlling the CT device 33 and for calculating sectional images or volume images of the examination object 39. In particular, the processing unit PRVS can be configured for controlling the temperature regulating unit, in particular, the fluid providing unit FLB and/or the heat exchanger W and/or the dehumidifier H, in particular, dependent upon the current temperature. An input facility 47, for example, a keyboard and an output apparatus 49, for example, a screen and/or a display can be connected to the processing unit PRVS, in particular, for signal purposes. The input unit 47 can advantageously be integrated into the output apparatus 49, for example, in the case of an, in particular, resistive and/or capacitive input display.

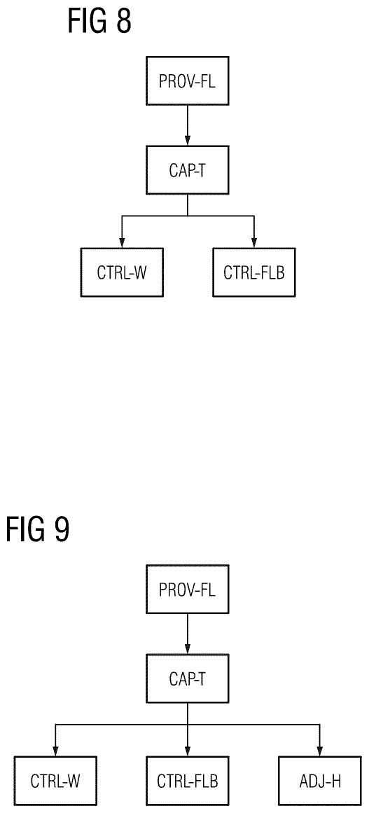

FIG. 8 shows a schematic representation of an advantageous embodiment of a proposed method for temperature regulation of a detector unit D of an X-ray detector 1. In a first step, the fluid can be provided PROV-FL via the fluid providing unit FLB to flow through the at least one fluid channel FLK. Therein, heat can be transferred between the detector unit D and the fluid. In a further step, a current temperature of the fluid and/or of the detector unit D and/or a currently provided fluid quantity is captured CAP-T via a sensor S. In dependence upon the captured current temperature and/or the captured currently provided fluid quantity, the heat exchanger W can be controlled CTRL-W such that the fluid is temperature-regulated to a pre-defined temperature or a pre-defined temperature range. Alternatively or additionally, dependent upon the captured current temperature, the fluid providing unit FLB can be controlled CTRL-FLB such that a fluid quantity provided per unit time is adjusted.

FIG. 9 shows a schematic representation of a further advantageous embodiment of a proposed method for temperature regulation of a detector unit D of an X-ray detector 1. Therein, the fluid can comprise a gas mixture. Furthermore, a water content in the fluid can be adjusted ADJ-H via a dehumidifier H before it flows through the at least one fluid channel FLK, in such a way that a dew point temperature of the fluid lies below the current temperature of the fluid.

The schematic representations contained in the drawings described do not represent any scale or size relationships.

Finally, it should again be noted again that the methods described above in detail and the apparatuses disclosed are merely exemplary embodiments which can be modified by a person skilled in the art in a wide variety of ways without departing from the scope of the invention. Furthermore, the use of the indefinite article “a” or “an” does not preclude the possibility that the relevant features can also be present plurally. Similarly, the expressions “unit” and “element” do not preclude the components in question consisting of a plurality of cooperating subcomponents which can possibly also be spatially distributed.

The expression “based upon” can be understood in the context of the present application, in particular, in the sense of the expression “using”. In particular, a formulation according to which a first feature is generated (alternatively: established, determined, etc.) based upon a second feature does not preclude the first feature being able to be generated (alternatively: established, determined, etc.) based upon a third feature.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, terms all (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program or code, instructions, some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible language), markup (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for flash example memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. An X-ray detector, comprising:
at least one detector unit; and
a temperature regulating unit including a fluid providing unit, a heat exchanger and at least one fluid channel, the fluid providing unit configured to provide a fluid, the heat exchanger configured to regulate a temperature of the fluid to a pre-defined temperature or a pre-defined temperature range, and the at least one fluid channel defining an area for the provided temperature-regulated fluid to flow through the at least one fluid channel, wherein
the detector unit and the temperature regulating unit are positioned in a defined arrangement relative to one another and are moveable relative to an examination object to be mapped via X-ray transirradiation,
the detector unit is configured to detect X-ray radiation incident upon an X-ray sensitive surface of the detector unit, and
the at least one fluid channel is arranged such that heat is transferrable between the detector unit and the fluid.

2. The X-ray detector of claim 1, wherein the detector unit is configured for photon-counting detection of the X-ray radiation incident upon the X-ray sensitive surface of the detector unit.

3. The X-ray detector of claim 1, wherein the temperature regulating unit includes a heating element configured to heat the detector unit to a further pre-defined temperature.

4. The X-ray detector of claim 1, wherein
the fluid comprises a gas mixture, and
the temperature regulating unit includes a dehumidifier configured to adjust a water content in the fluid before the fluid flows through the at least one fluid channel, such that a dew point temperature of the fluid lies below a current temperature of the fluid.

5. The X-ray detector of claim 1, wherein
the fluid comprises a gas mixture,
the fluid providing unit is configured to provide the fluid from a first environment to the heat exchanger,
the temperature regulating unit is configured to discharge the fluid, after an exchange of the heat with the detector unit, from the at least one fluid channel into a second environment.

6. The X-ray detector of claim 1, wherein the fluid providing unit is configured to provide the fluid, after an exchange of the heat with the detector unit, from the at least one fluid channel repeatedly to the heat exchanger.

7. The X-ray detector of claim 1, wherein
the fluid providing unit comprises at least one of a pump, a blower, or a nozzle, and
the at least one of the pump, the blower or the nozzle is configured to provide the fluid at a pre-defined pressure.

8. The X-ray detector of claim 1, wherein
the detector unit comprises a plurality of detector modules
the temperature regulating unit comprises at least one fluid channel to each of the plurality of detector modules,
the fluid channels are able to have the fluid flow through the fluid channels, and
the fluid channels are arranged such that the heat is transferable between the detector modules and the fluid.

9. The X-ray detector of claim 8, wherein the fluid providing unit comprises a subsidiary fluid providing unit to each of the detector modules, each subsidiary fluid providing unit is configured to provide the temperature-regulated fluid to the respective fluid channel.

10. The X-ray detector of claim 9, wherein
the X-ray detector further comprises a sensor configured to capture at least one of a current temperature of at least one of the fluid or the detector unit or a currently provided fluid quantity, and
the heat exchanger is configured to regulate the temperature of the fluid based on the at least one of the current temperature, the provided fluid quantity or wherein the fluid providing unit is configured to adjust a provided fluid quantity per unit time based on the current temperature.

11. The X-ray detector of claim 10, wherein the subsidiary fluid providing units are configured to adjust a respectively provided fluid quantity per unit time based on the current temperature at least one of individually or in a coordinated manner.

12. The X-ray detector of claim 1, wherein the heat exchanger is configured to regulate the temperature of the fluid to a temperature between 18° C. and 30° C.

13. An X-ray device comprising:
an X-ray source; and
the X-ray detector of claim 1, wherein the X-ray source is configured to emit X-ray radiation for transilluminating an examination object arranged between the X-ray source and the X-ray detector.

14. The X-ray device as claimed in claim 13, wherein the X-ray source and the X-ray detector are mounted in a defined arrangement that is rotatable about a common rotation axis.

15. A method for temperature regulating a detector unit of the X-ray detector of claim 1, comprising:
providing the fluid via the fluid providing unit to flow through the at least one fluid channel, wherein the heat is transferred between the detector unit and the fluid;
capturing at least one of a current temperature of the at least one of the fluid or the detector unit, or a currently provided fluid quantity, via a sensor; and
at least one of
controlling the heat exchanger based on the at least one of the captured current temperature or the captured current fluid quantity such that the fluid is temperature-regulated to the pre-defined temperature or the pre-defined temperature range, or
controlling the fluid providing unit based on the captured current temperature such that a provided fluid quantity per unit time is adjusted.

16. The method of claim 15, wherein
the fluid comprises a gas mixture,
a water content in the fluid is adjusted via a dehumidifier before it flows through the at least one fluid channel such that a dew point temperature of the fluid lies below the current temperature of the fluid.

17. The X-ray detector of claim 3, wherein
the fluid comprises a gas mixture, and
the temperature regulating unit includes a dehumidifier configured to adjust a water content in the fluid before the fluid flows through the at least one fluid channel, such that a dew point temperature of the fluid lies below a current temperature of the fluid.

18. The X-ray detector of claim 17, wherein the fluid providing unit is configured to provide the fluid, after an exchange of the heat with the detector unit, from the at least one fluid channel repeatedly to the heat exchanger.

19. The X-ray detector of claim 18, wherein
the fluid providing unit comprises at least one of a pump, a blower, or a nozzle, and
the at least one of the pump, the blower or the nozzle is configured to provide the fluid at a pre-defined pressure.
